(19) United States
(12) Patent Application Publication
(10) Pub. No.: US 2007/0167994 A1
(43) Pub. Date: Jul. 19, 2007

(54) Telemetry method and apparatus for ambulatory medical device

(75) Inventors: Brian M. Shelton, Northridge, CA (US); Sam W. Bowman, Valencia, CA (US)

Correspondence Address: HENRICKS SLAVIN AND HOLMES, LLP;ADVANCED BIONICS CORPORATION, 840 APOLLO STREET, SUITE 200, EL SEGUNDO, CA, 90245, US

(73) Assignee: ADVANCED BIOICS CORPORATION, Valencia, CA (US)

(21) Appl. No.: 10/596,311

(22) PCT Filed: Nov. 17, 2004
(86) PCT No.: PCT/US04/38560

§ 371 Date: Jun. 8, 2006

Related U.S. Application Data
(60) Provisional application No. 60/529,277, filed on Dec. 12, 2003.

(51) Int. Cl. A61N 1/00

(52) U.S. Cl. 607/60

(57) ABSTRACT

A medical system including an implantable battery operated therapy device and an external communication device configured for wireless communication in a manner to minimize energy consumption in the implanted device. The medical device is configured to wake periodically from a sleep state to look for a message preamble from the communication device. The preamble is formatted to include control information, e.g., information indicating the time of receipt of the forthcoming message.

### FIELD OF THE INVENTION

This invention relates generally to medical systems which include at least one ambulatory medical device, a communication device, and wireless telemetry means for communicating data and control information between said communication device and said medical device.

### BACKGROUND OF THE INVENTION

Various medical systems are described in the prior art which include wireless telemetry subsystems for communicating data and control information between a communication device (CD), e.g., an external controller operable by a physician or patient, and an ambulatory medical device (MD), e.g., an implantable infusion pump.

Inasmuch as typical ambulatory medical devices, and particularly implantable medical devices, are battery powered, it is desirable to configure and operate such devices so as to minimize energy consumption therein in order to extend device life and reduce the risk associated with surgical replacement. As recognized in U.S. Pat. No. 6,427,088, whose disclosure is incorporated herein by reference, telemetry reception and transmission operations are typically highly energy consumptive and accordingly, it is preferable to minimize the occurrence and duration of such telemetry operations.

In order to minimize telemetry operation duration, some prior art systems only actuate the medical device (MD) telemetry subsystem in response to some distinct occurrence, such as when an external magnet is brought into physical proximity with a reed switch in the medical device. In other prior art systems, the MD telemetry subsystem normally operates in a low energy consuming state and periodically wakes to define a “listening period”, during which it looks for an incoming message from the communication device (CD). Because such an MD telemetry subsystem only wakes periodically, the CD telemetry subsystem typically transmits a message “preamble” before each message to alert the medical device of the forthcoming message and cause the MD telemetry subsystem to stay awake until the message arrives. (As used herein, “preamble” refers to that part of a transmission that allows a receiver to find (or synchronize to) the beginning of the message. As such, it is generally not necessary to receive a preamble in its entirety in order to receive the message. The “preamble” is distinguishable from the “message” in that the message is that part of the transmission that must be received in its entirety in order for the receiver to properly respond to the message). Such a system typically requires that the CD telemetry subsystem transmit a preamble of sufficient length to span the time between successive listening periods. Unfortunately, in such systems, the medical device can consume more energy in receiving the preamble than it does in receiving the message.

A typical message preamble format comprises a repeating predetermined bit pattern which the medical device can readily distinguish from noise. Column 22 of aforementioned U.S. Pat. No. 6,427,088 discusses exemplary bit patterns and other aspects of preambles used for communications from the communication device to the medical device and also for communications from the medical device to the communication device.

Other schemes have been proposed in the prior art to increase the energy efficiency of an ambulatory medical device. One such scheme tracks the drift between the clocks of the medical device and the communication device to enable the communication device to predict when the medical device will wake to look for a message preamble. The communication device uses this drift information together with known periodicity of the medical device to begin preamble transmission immediately before the medical device wakes so that it need only transmit preamble for a short interval to establish communication between the two devices.

Another prior art scheme which tracks clock drift grows the length of the preamble in relation to the amount of elapsed time since the last successful communication. In such a scheme, the communication device synchronizes its clock with the medical device when a successful communication occurs. When the communication device again attempts to communicate, it sets its preamble length to a length that is proportional to the amount of time that has passed since the last communication.

### SUMMARY OF THE INVENTION

The present invention is directed to energy conservation enhancements useful in medical systems of the type including an ambulatory medical device (MD) and a communication device (CD) having wireless telemetry means for communicating data and control information between the devices.

In accordance with a significant aspect of the present invention, a preamble transmitted by the communication device to alert the medical device of a forthcoming message carries embedded control information. The control information is used to reduce the time duration during which the medical device must listen, thus minimizing energy consumption in the medical device. More particularly, an object of the invention is to minimize the time duration that the MD telemetry subsystem is in a high energy consumptive awake (or “signal receiving” or “listening”) state and maximize the time that it is in its low energy consumptive sleep state.

In a preferred embodiment, the embedded control information predicts the time duration to the next message, thereby enabling the MD telemetry subsystem to switch to a low energy consumptive state for an interval related to the predicted time duration.

In an alternative embodiment useful in a system including multiple addressable medical devices, the embedded control information is used to identify the intended destination medical device for a forthcoming message. This information enables all devices other than the destination device to remain in a low energy consumptive state.

Various encoding schemes can be used in accordance with the invention to embed control information in the preamble. The encoding can be explicit or implicit. For example, one approach would be for the communication device to simply periodically insert a “time to next message” word in the preamble bit stream. It is preferable, however, to implicitly integrate the control information into the preamble in a manner which minimizes an increase in bit overhead. That is, it is desirable to minimize the number of extra bits which need to be introduced to encode the control information.

In accordance with a preferred encoding scheme, the preamble comprises a serial bit stream formed of successive N bit words, e.g., 8 bit words, where each word includes a most significant, i.e., highest order, bit of constant binary value (1 or 0) and a least significant, i.e., lowest order, bit which toggles for each successive word. This scheme enables word boundaries to be derived from the bit stream by performing an exclusive-OR operation with respect to two successive words. The operation results in the most significant bits always producing a 0 and the least significant bits always producing a 1. Thus, adjacent most significant and least significant bits produce a reliable bit transition. By assuring that this transition is unique, a word boundary can be determined and the embedded control information can be extracted from the bit stream.

In a preferred embodiment of the invention, the preamble is comprised of a monotonic sequence of words representing counts of a decrementing (or incrementing) counter counting toward some predefined terminating count value (e.g. zero). That is, each successive word represents a count value, or duration, and is succeeded by a word having a count value equal to that of the prior word minus one. Thus, each count value extracted by the medical device from a received preamble bit stream will predict the time duration to the terminating count value. This time indicates the arrival time of the next message. The unique bit transition is guaranteed, because the exclusive-OR (XOR) of any two consecutive integers contains one and only one bit transition from 0 to 1 as this XOR result is read from the most significant bit to the least significant bit. If a full count sequence is shorter than the length of the preamble, it can be repeated. In this situation, the MD telemetry subsystem would typically wake for each repetition.

In a similar but alternative configuration, the communication device can embed a master count between each subsequence, i.e., one cycle of a full count sequence. In this situation, the medical device telemetry subsystem wakes for each subsequence to receive enough control information to enable it to determine the current master count and count value in the subsequence.

In alternative embodiments, the words of the message preamble can carry other types of control information. For example, in a medical system including multiple medical devices, each word can additionally or alternatively identify the particular medical device intended as the destination for the forthcoming message. This scheme can conserve energy by reducing wake-up frequency of the non-addressed medical devices.

### BRIEF DESCRIPTION OF THE FIGURES

FIG. 1 is a block diagram of an exemplary medical system comprised of one or more ambulatory medical devices, e.g., an implantable infusion pump, and an external communication device, e.g., a controller operable by a clinician and/or patient for programming each medical device;

FIG. 2 is a timing diagram depicting a typical prior art procedure for operating the medical system of FIG. 1 wherein the external communication device transmits a message preamble to the medical device which then stays awake until the message is received;

FIG. 3 is a timing diagram depicting a system in accordance with the present invention wherein the preamble includes control information recognizable by the medical device to enable the medical device to sleep until just before the message arrives;

FIG. 4 is a chart depicting a typical preamble serial bit stream in accordance with the invention and a procedure for extracting a duration count value from the bit stream;

FIG. 5 is a flow chart depicting an algorithm executable by the medical device processor for processing the preamble bit stream in accordance with the procedure depicted in FIG. 4;

FIG. 6 is a timing diagram similar to FIG. 3 but depicting one alternative embodiment of the invention; and

FIG. 7 is a timing diagram similar to FIG. 3 but depicting a second alternative embodiment of the invention;

FIG. 8 is a chart depicting the same exemplary preamble bit stream as in FIG. 4 and showing how the bit stream can be processed to distinguish the preamble from noise;

FIG. 9 is a flow chart depicting an algorithm executable by the medical device processor for processing the preamble bit stream in accordance with the procedure depicted in FIG. 8;

FIG. 10 is a chart substantially identical to FIG. 4 but representing a procedure useful for extracting constant information that is embedded in the preamble, such as a device identification code, rather than variable information, such as a duration count value; and

FIG. 11 is a flow chart depicting an algorithm executable by the medical device processor for processing the preamble bit stream in accordance with the procedure depicted in FIG. 10.

### DETAILED DESCRIPTION

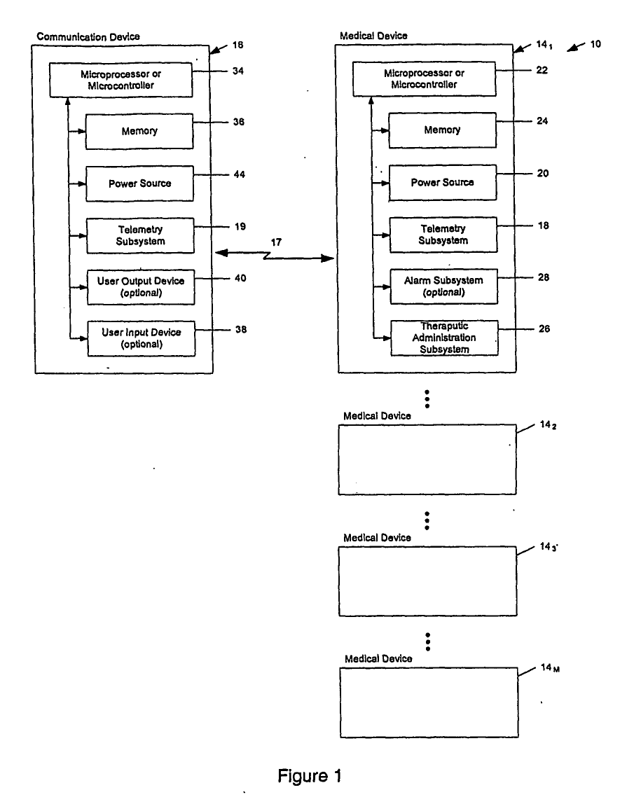

Attention is initially directed to FIG. 1 which comprises a generalized block diagram of a medical system 10 of the type which can advantageously employ the teaching of the present invention. The system 10 is basically comprised of one or more ambulatory medical devices (MD) 14 (e.g., 14₁, 14₂, . . . 14_{M}) and a communication device (CD) 16. The system of FIG. 1 contemplates that the communication device 16 and the medical devices 14 communicate wirelessly, e.g., via RF telemetry 17 using MD telemetry subsystem 18 and CD telemetry subsystem 19 respectively contained with the devices 14 and 16. Each of the ambulatory medical devices 14 can be configured for use internal or external to a patient's body. However, in the more significant applications of the present invention, each medical device 14 is implanted in a patient's body to perform some therapeutic function, such as controlled drug delivery or nerve stimulation. The communication device 16, on the other hand, is primarily intended to be deployed external to the body and available for use by a physician or clinician or patient to generate control and data signals for transmission to a medical device 14. For example, using the communication device 16, a clinician is able to produce command signals which are transmitted via RF link 17 to a medical device 14 to affect its therapeutic performance such as by modifying its drug delivery profile. Systems of the type thus far described are well known in the prior art, e.g., aforementioned U.S. Pat. No. 6,427,088. The present invention is particularly directed to a method and apparatus configured to more efficiently operate the system 10 of FIG. 1 to minimize energy consumption in each of the medical devices 14.

More particularly, each medical device 14 in system 10 typically includes an internal power source 20, e.g., a battery. It is desirable in accordance with the present invention to operate each device 14 to minimize energy consumption so as to maximize the useful life of the power source 20 and reduce the need for surgical replacement of the device 14 and/or source 20.

A typical medical device 14 includes an MD microprocessor (or microcontroller) 22 having a memory 24 associated therewith for storing programs and data. The microprocessor 22 operates to execute a stored program to control MD subsystem 26 to administer some type of therapy; e.g., drug delivery, nerve stimulation, etc. Each medical device 14 may also include an MD alarm subsystem 28 controllable by microprocessor 22 to alert the patient or clinician of some monitored event. Microprocessor 22 also controls the operation of MD telemetry subsystem 18 to selectively place it in an awake (signal receiving) state or a sleep, low energy consuming state.

Communication device 16 is shown as including a CD microprocessor (or microcontroller) 34 which operates in conjunction with memory 36 which stores programs and data. The device 16 optionally includes a CD user input device 38, e.g., a keyboard and a CD user output device 40, e.g., a display. The communication device 16 further includes aforementioned CD telemetry subsystem 19 configured to transmit signals to or receive signals from the MD telemetry subsystem 18. The communication device 16 includes an internal power source 44 which can comprise a battery or any other suitable conventional power source.

In a typical system 10, the communication device 16 is capable of sending messages to an intended destination medical device 14 for use by its microprocessor 22 to affect the operation of its therapeutic administration subsystem 26. Additionally, each medical device 14 is capable of sending messages to the communication device 16 to report various conditions, e.g. battery status, drug reservoir status, etc. These respective messages sent by the communication device 16 and medical device 14 are handled by the respective telemetry subsystems 19 and 18, each of which is able to transmit and receive RF telemetry signals. Typically, these RF telemetry signals comprise bit streams carried by an RF carrier signal of specified frequency. In addition to message transmissions initiated by either the communication device 16 or medical device 14, each device typically responds to a received message by generating an appropriate RF telemetry acknowledgement signal.

In a typical application of the system of FIG. 1, a patient having a medical device 14 implanted in his body may visit a physician every 1-3 months for a checkup. The physician may desire to modify the therapy being administered by the implanted medical device and accordingly can operate the communication device user input 38 to transmit a message via subsystem 19 to the telemetry subsystem 18 of a medical device 14. Inasmuch as such transmissions, in typical use, occur relatively infrequently, e.g., monthly, and because the telemetry subsystem 18 is generally relatively highly energy consumptive when operating in a signal receiving mode, energy conservation techniques have been developed to keep medical device subsystem 18 in a sleep, i.e., low energy consumptive state, for as long as possible.

For example, in some prior art systems, the telemetry subsystem 18 is normally maintained in its sleep state and is awakened periodically by microprocessor 22 for short listening periods (or windows) to look for an incoming message from communication device 16. Because the telemetry subsystem 18 normally only wakes periodically for short durations, the communication device 16 is typically designed to transmit a preamble signal having a predetermined format (known to both the medical device and communication device) prior to transmitting a message signal. The preamble is intended to alert the subsystem 18 that a message will soon arrive. If the medical device 14 during one of its periodic listening periods recognizes the preamble, then it stays awake until the message is received. In such systems, the preamble must be sufficiently long to span the sleep period between successive periodic listening periods in order to assure that the medical device is alerted to a forthcoming message. To enable the, medical device to recognize the preamble and readily distinguish it from noise, the preamble format is typically comprised of a repeating specific bit pattern.

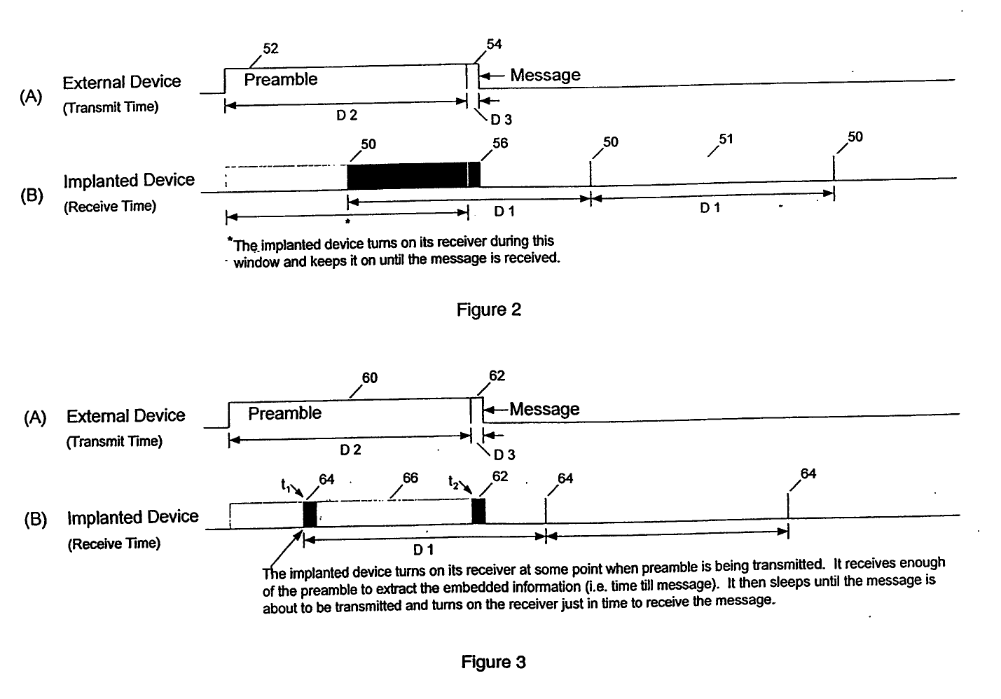

FIG. 2, which diagrammatically depicts the operation of such a prior art system, shows in line (B) that the medical device, or “implanted device”, 14 defines short duration listening periods 50 spaced by relatively long duration (D1) sleep periods 51. Line (A) shows the preamble 52 transmitted by the communication device, or “external device”, 16 as spanning a duration D2. The duration D2 should be at least as long as the duration D1 of a sleep period 51. The message 54, shown as having a duration D3 immediately follows the preamble 52. In operation, a listening period 50 can, of course, occur anywhere within the preamble 52 and accordingly in order to assure receipt of the following message 54, the device 14 is shown as staying awake until the message 54 is received (shown at 56). It is typical for a periodic listening period duration to be very short, e.g., on the order of 20 milliseconds, with the sleep periods 51 between successive listening periods having a much longer duration, e.g., on the order of 5 seconds. Accordingly, a considerable amount of energy can be expended in the device 14 while it is maintained in the awake state awaiting for receipt of a message 54.

Other prior art systems attempt to reduce this inefficient use of energy by configuring tie device 16 to predict the time occurrence of the next listening period. Such schemes, for example, which may require that the device 16 track the drift between respective clocks in the devices 14 and 16, are relatively complex and, at best, have met with only limited success.

The present invention is directed to an improved energy conservation system in which control information is embedded in the preamble for use by the MD microprocessor 22 to more efficiently control the MD telemetry subsystem 18. In a preferred embodiment of the invention, the embedded control information communicates the time duration to arrival of the next message. This control information can be read by the medical device 14 during a periodic listening period to enable the device to return to a low energy consuming sleep state for an interval approximately equal to the indicated time duration to the next message. This scheme is represented in FIG. 3 where line (A) shows the preamble 60 transmitted prior to the message 62. Line (B) shows a periodic listening period 64 occurring at time t₁ during the preamble. During this listening period 64, the device 14 reads enough embedded control information to enable it to go back to sleep for the interval 66 and then wake at time t₂ to receive the message 62 (including any appropriate bit, byte, and frame synchronization signals). In this embodiment, the duration D2 should be at least as long as the duration D1 plus the amount of time required to power up the receiver and receive two of the count values contained within the preamble.

Prior art systems are able to distinguish preamble from noise by verifying to verify that the received bits match the expected repeating bit pattern. Embodiments of the present invention cannot use the same method, because the specific information contained within the preamble is not known to the receiver prior to reception. However, the preamble format of the present invention includes more subtle characteristics which allow the preamble to be distinguished from noise and which can be recognized in a suitable mathematical or algorithmic manner. In accordance with the preferred embodiment, the preamble comprises a serial bit stream formed of successive N bit words, e.g., 8 bit words, where each word contains a count value indicative of the time-to-arrival of the next message. For simplicity herein, a word shall be assumed to comprise an 8 bit word but it should be understood that words of different lengths can be used in accordance with the invention.

In accordance with the preferred embodiment, a counting scheme is employed in which the preamble is comprised of a monotonic sequence of words where each word represents a count value. Consider, for example, the following sequence of three successive counts of a decrementing counter

$$\underline{MS} … \underline{LS} \\ ( Ct 1 ) 01010000$$

$$( Ct 2 ) 01001111$$

$$( Ct 3 ) 01001110$$

Note that the binary value of the least significant (LS) bit toggles between each successive count and that the binary value of the most significant (MS) bit stays constant.

In accordance with a preferred embodiment of the present invention, the binary value of the most significant (MS) bit is maintained constant (shown as 0 in the above exemplary sequence) to enable word boundaries to be extracted from the preamble which comprises a serial bit stream of contiguous words, as will be explained hereinafter

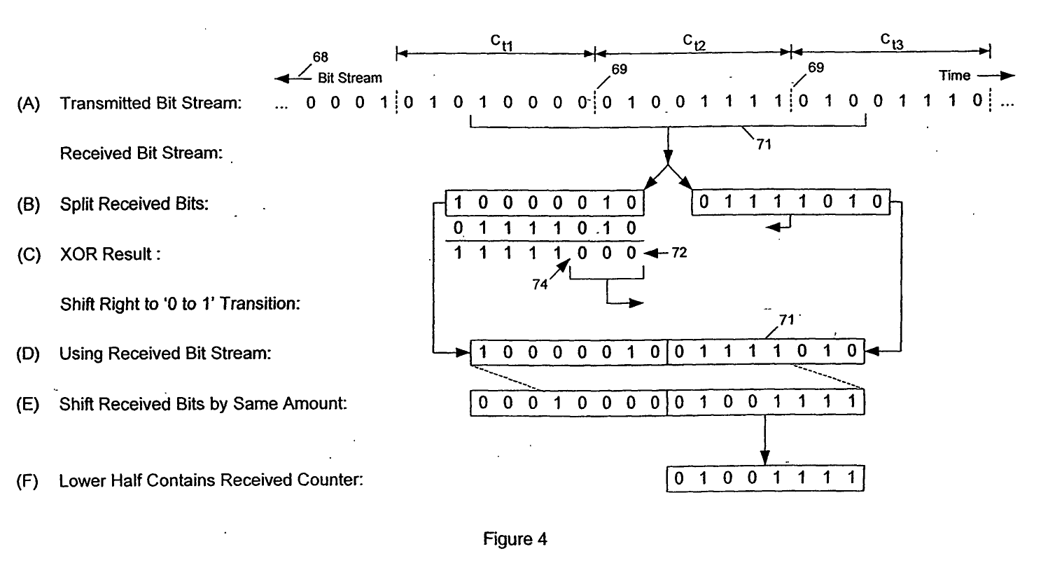

FIG. 4 in line (A) represents a preamble bit stream 68 containing the above exemplary count sequence (Ct1, Ct2, Ct3 . . . ) with the bits assumed to be moving from right to left and with each word being transmitted most significant bit first. To determine the count value of each word, the word boundary, i.e., beginning and end of each word must be identifiable in the bit stream. Although this could be accomplished by introducing special bit sequences into the stream, it is preferable to avoid the overhead of extra bits. Accordingly, in a preferred embodiment, a word boundary is reliably determined by looking for a unique transition in the stream which will occur as a consequence of the aforementioned formatting, i.e., LSB=toggle bit and MSB=constant. The position of this transition is determined in accordance with a preferred implementation by executing an XOR operation with respect to successive N bit groups. The XOR operation always produces a 0 with respect to the constant most significant bits and always produces a 1 with respect to the toggling least significant bits. This reliable 1-0 transition enables the boundary position 69 between successive words to be located and thus enables a count value to be extracted from the bit stream.

More particularly, a preferred scheme for processing the preamble bit stream 68 to locate a word, or count value, boundary 69 is depicted in FIG. 4. Initially, a 2N bit grouping from the stream is captured (e.g., 1000001001111010) in sample window 71 and split into an upper N bit group (i.e., 10000010) and a lower N bit group (i;e., 01111010) as represented in line (B). These upper and lower groups are then exclusively ORed to produce the N-bit result 72 shown in line (C) where Result bit 0 (LSB)=0 and Result bit N (MSB)=1. Note that the result 72 includes a single bit transition 74 occurring between Result bits 2 and 3. This transition position, in fact, corresponds to the aforementioned word boundary between the least significant toggle bit of one word and the most significant constant bit of the following word. In the example depicted in FIG. 4, this transition 74 is located three bits left of the least significant bit in result 72. This means that the originally selected 2N bit sample (line (B)) is in fact displaced from word boundaries in the preamble stream 68 by three bits. Accordingly, the 2N bit grouping captured in window 71 can be shifted right three bits (line (E)) so that its lower half (line F) will then constitute a bit stream which contains a count value which predicts the time duration to the next message.

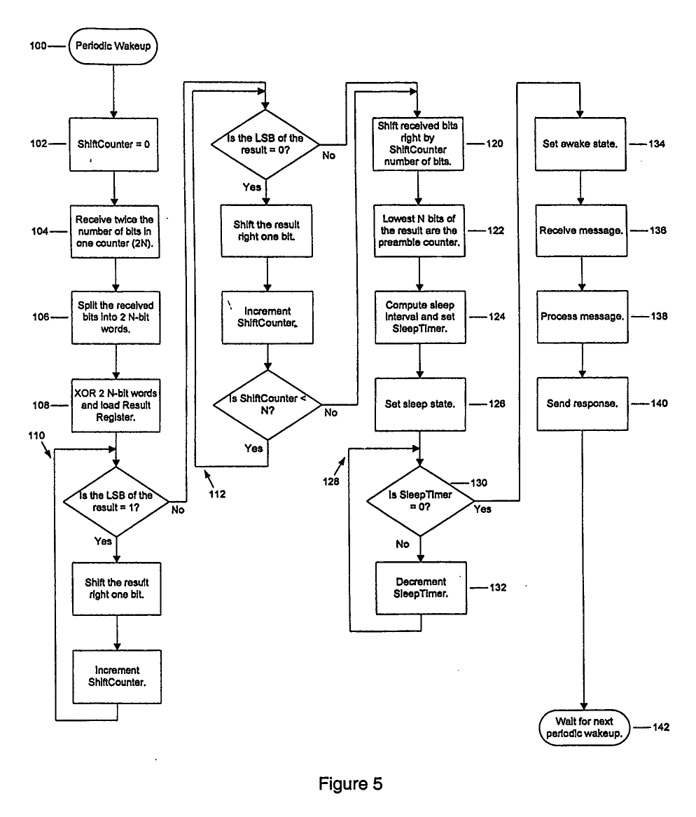

FIG. 5 comprises a flow chart of a program executable by MD microprocessor 22 for performing the count value extraction operation represented in FIG. 4. Block 100 represents a periodic waking of the MD telemetry subsystem 18 to define a listening period. Block 102 initializes the ShiftCounter by setting it to zero. Block 104 represents the step of capturing the 2N bit grouping depicted in window 71 of line (A) of FIG. 4. Block 106 represents the step of splitting the 2N bit grouping into upper and lower N bit groups depicted in line (B) of FIG. 4. Block 108 represents the XOR operation performed on the upper and lower N bit groups to produce the result depicted in line (C) of FIG. 4.

After execution of block 108, loops 110 and/or 112 are executed to locate the position of the transition 74 (FIG. 4) within the N bit result of the XOR operation. Loop 110 is executed if the least significant bit of the result is 0 whereas loop 112 is executed if the least significant bit of the result is 1. Each iteration through either loop increments the aforementioned ShiftCounter. The resulting value in the ShiftCounter determines the number of bits that the captured 2N bit grouping must be right shifted to extract the count value, as was depicted in lines D, E, F of FIG. 4. This right shifting operation is represented by block 120 in FIG. 5. Block 122 represents the step of using the low order N bits as the extracted preamble word containing the sought control information, i.e., count value (depicted in FIG. 4, line (F)). Thus loops 110 and 112, together with blocks 120 and 122 constitute the procedure for extracting a count value from the preamble stream.

Block 124 uses the count value acquired in block 122 to calculate a sleep interval, i.e., the time duration that the subsystem 18 can sleep before having to wake to receive the message. Most simply, the calculation is merely the product of the count value and some constant which depends on the systems' timing characteristics. This calculated interval is set into a SleepTimer in block 124 and the subsystem 18 resumes its sleep state in block 126.

The MD microprocessor 22 then executes loop 128 comprised of decision block 130 which asks whether the SleepTimer equals zero and operation block 132 which decrements the SleepTimer. When decision block 130 determines that the SleepTimer does equal zero, operation branches to block 134 which sets the subsystem 18 to its awake state in time to receive the message following the preamble. That is, the message is received in block 136, processed in block 138, and acknowledged in block 140. Then, as represented by block 142, the subsystem 18 resumes it's sleep state, waiting for its next periodic wakeup. Alternatively, loop 128 could be implemented with a hardware timer.

As previously mentioned, in the scheme depicted in FIG. 3, if a full count sequence is shorter than the required preamble duration, the sequence can be repeated. FIG. 6 illustrates such a repeating sequence scheme in which the CD telemetry subsystem 19 successively transmits preamble count sequences N . . . 2, 1 spanning a time duration D1, prior to transmitting the message 150. The MD telemetry subsystem 18 wakes for a periodic listening period 152 (which is random relative to the preamble) during an initial count sequence N to enable MD processor 22 to extract the embedded count value representing the time duration to the end of that count sequence. The subsystem 18 can then return to the sleep state and thereafter wake just after the completion of each subsequent count sequence for a listening period 154 to determine whether a preamble or a message is being transmitted.

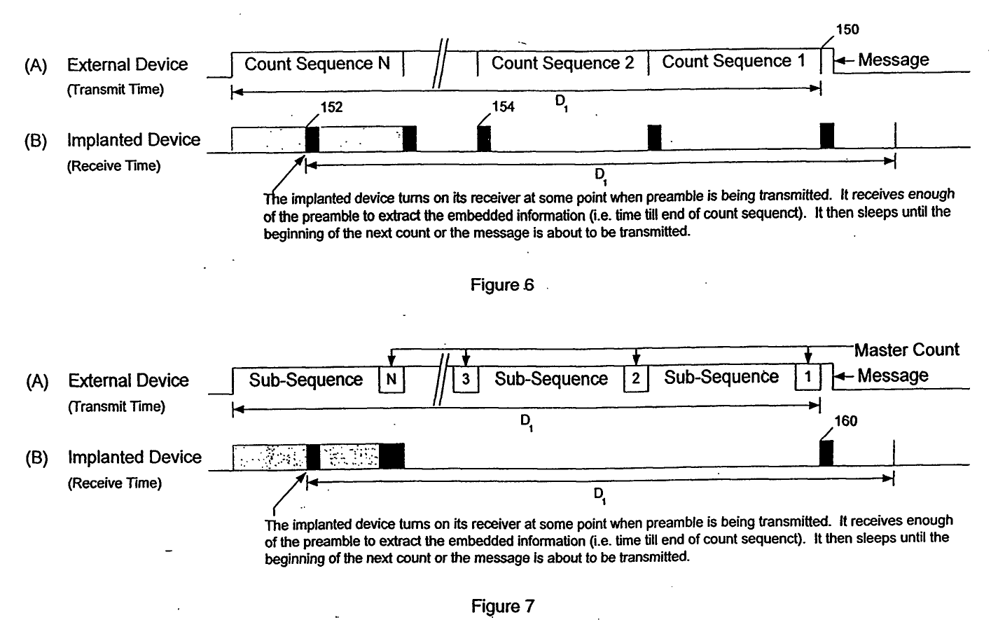

A variation of the repeating sequence scheme of FIG. 6 is illustrated in FIG. 7 which contemplates that the communication device 16 embed a master count (N . . . 2, 1) between each sequence repetition. In this implementation, the subsystem 18 wakes for a periodic listening period (during master count N) to enable the MD microprocessor 22 to extract enough of the preamble to determine the master count and where it is in the preamble sequence. The MD microprocessor computes the sleep time by multiplying the extracted master count by the amount of time that is required to transmit one full cycle of the sub-sequence. This transmission time can be known either a-priori or by extracting the first count of the sub-sequence after the master count. After computing the sleep time, the subsystem 18 sleeps until it is time to awaken and receive the message 160.

The operation represented in FIG. 5 demonstrated how a count value can be extracted from the preamble stream on the assumption that the preamble has been recognized. It should be appreciated that when information is embedded in the preamble in accordance with the invention, as contrasted with a prior art preamble comprising a simple repeating bit pattern, it becomes more difficult for the MD telemetry subsystem 18 to distinguish the preamble from noise. In order to facilitate preamble recognition, the number of bit transitions occurring in the N bit result of the aforementioned XOR operation can be counted. With the preamble counter format discussed in FIG. 4, the XOR operation result will contain one bit transition if the sample window 71 is aligned with the words in the bit stream and either two or three transitions if misaligned. There are only seven possible bit transitions in the XOR result. These are the seven between each of the eight bits. However, when the number of bit transitions is odd, the MSB and LSB will not match. If these ‘wrap-around’ bits are treated like a possible transition, the number of valid transitions in a counting preamble is either two or four. Any number of transitions other than two or four is either a result a corrupted preamble or no preamble being transmitted.

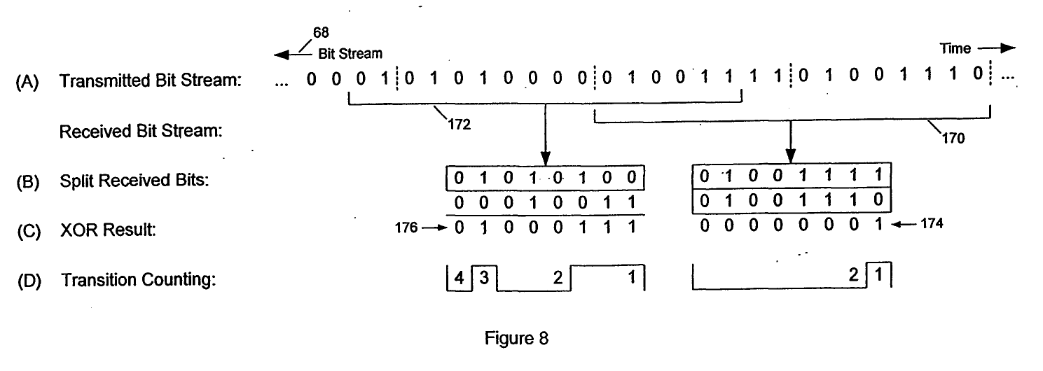

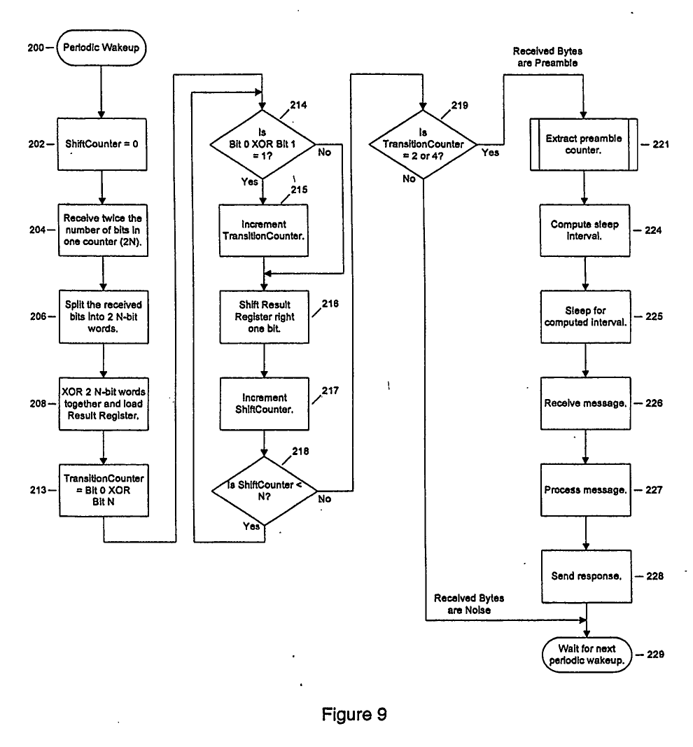

FIG. 8 shows the same bit stream 68 shown in FIG. 5 and depicts in line (A) two sample windows 170, 171, each of which captures a 2 N-bit sample. Each sample is split into an upper N bit group and a lower N bit group as shown in line (B). The upper and lower N bit groups are then XORed to produce the N-bit results shown in line (C). Line (D) shows the result 174 attributable to the sample captured in window 170 which happens to be aligned with the word boundaries in stream 68. Note that the result 174 is comprised of two bit transitions, i.e., from bit 0 (LSB) to bit 1 and from bit 7 (MSB) to bit 0.

Line (D) also shows the result 176 attributable to the sample captured in window 172 which is misaligned with the word boundaries in stream 68. Note that the result 176 is comprised of four bit transitions, i.e., from bit 2 to bit 3, from bit 5 to bit 6, from bit 6 to bit 7 and from bit 7 to bit 0.

Indeed, every N-bit result produced by the XOR operation depicted in FIG. 8 will consist of two bit transitions if the sample window is aligned with the words contained within the bit stream or two or four bit transitions if the sample window is misaligned. This characteristic can be used by the MD microprocessor to distinguish preamble from noise. That is, a received signal will be interpreted as preamble only if the XOR operation produces results having two or four bit transitions.

FIG. 9 is a flow chart similar to FIG. 5 but modified to show how transition counting (based on FIG. 8) can be incorporated to better enable the MD processor 22 to recognize the preamble. In FIG. 9, blocks 200, 202, 204, 206, 208 respectively correspond to blocks 100, 102, 104, 106, 108 of FIG. 5 and operate substantially identically. After execution of block 208, MD processor 22 executes blocks 213 through 219 to count the bit transitions in the XOR result (FIG. 8, line (c)) to determine whether the received signal constitutes preamble.

More particularly, block 213 executes an XOR operation to compare bit 0 and bit N (8) of the XOR result (FIG. 8, line (8)) to determine whether they are the same or different. If they are different (thus constituting a bit transition) block 213 sets a Transition Count to 1 and if they are the same, the Transition Count is set to 0. Block 214 similarly executes an XOR operation to compare bits 0 and 1. If they are different, the Transition Count is incremented in block 215. For simplicity, it is assumed that the XOR result being examined is stored in a Result Register and that the next two bits, i.e., bits 1 and 2 can next be compared by right shifting the Result Register (block 216) prior to again executing block 214. For every execution of block 216, a Shift Count is incremented (block 217). Decision block 218 asks whether the Shift Count is less than N. If yes, meaning that all of the XOR result bits have not yet been compared, then operation loops from block 218 back to block 214.

After decision block 218 determines that all of the result bits have been compared so that the Transition Count now indicates the number of transitions in the result, operation proceeds to decision block 219. Block 219 asks whether the Transition Count is 2 or 4. If not, the received signal must be interpreted as noise and operation branches to block 242 to await the next periodic wakeup. On the other hand, if the Transition Count is 2 or 4, then operation proceeds from block 219 to block 221 to extract the control information, i.e., count value, embedded in the preamble. Block 221 of FIG. 9 essentially corresponds to loops 110, 112 and blocks 120, 122 of aforementioned FIG. 5. Thereafter, operation proceeds through blocks 224 and 225 (where block 225 corresponds to blocks 126, 130, 132, and 134 of FIG. 5) and blocks 236, 238, and 240 (corresponding to blocks 136, 138 and 140 of FIG. 5).

In the specific embodiments described thus far, the control information embedded in the preamble has been assumed to comprise count values predicting the time duration to arrival of the next message. It has been mentioned, however, that the embedded control information can be used for other purposes to conserve energy in the medical device, such as an identification code (ID) to designate a particular one of multiple devices.

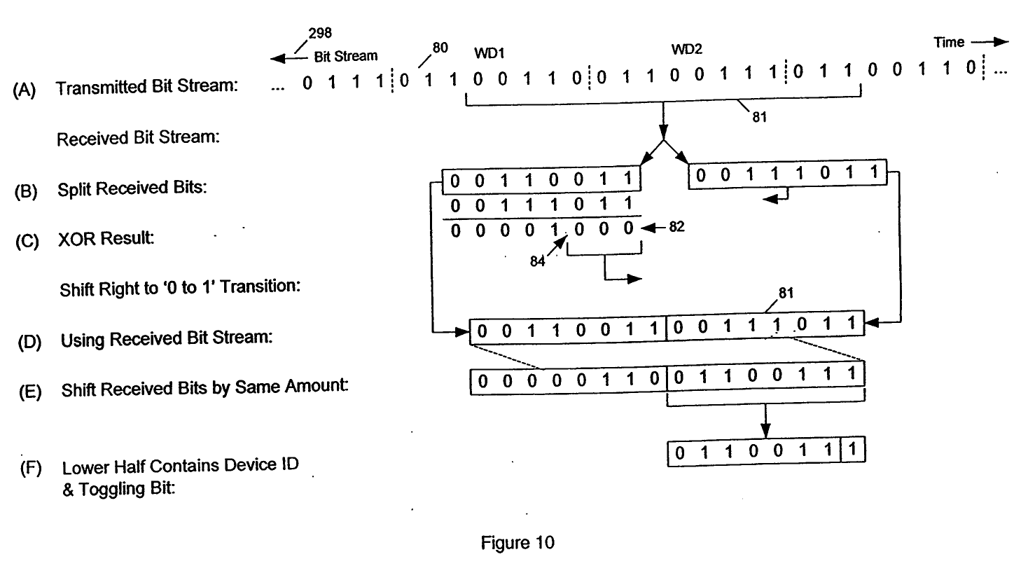

Line (A) of FIG. 10 shows an exemplary preamble bit stream 298 comprised of successive N bit words, assumed to be 8 bit words, where each word LSB (i.e., bit 0) comprises a toggle bit having a valve of 0 or 1 and where the M (7) more significant bits comprise an ID code. More particularly, note that word WD1 in stream 298 has a toggle bit having a value of 0 and WD2 has a toggle bit having a value of 1. The ID code in both WD1 and WD2 is depicted as 0110011. FIG. 10 depicts the scheme used to extract the control information, i.e., the ID codes from the bit stream 298.

It should be apparent that the scheme depicted in FIG. 10 is substantially identical to the extraction scheme depicted in FIG. 4. It differs in that in FIG. 4, the toggle bit (LSB) constitutes an integral part of the count value whereas in FIG. 10 the toggle bit is not a substantive part of the M (7) bit ID code but rather is appended to each M bit ID code to force the periodic transitions in the bit stream, as has been discussed. The procedure of FIG. 10 produces the extracted control information similarly to FIG. 4 but in FIG. 10, after the N bit word shown in line (F) is extracted, the toggle bit is dropped so as to leave the remaining M (7) bits as the extracted ID code.

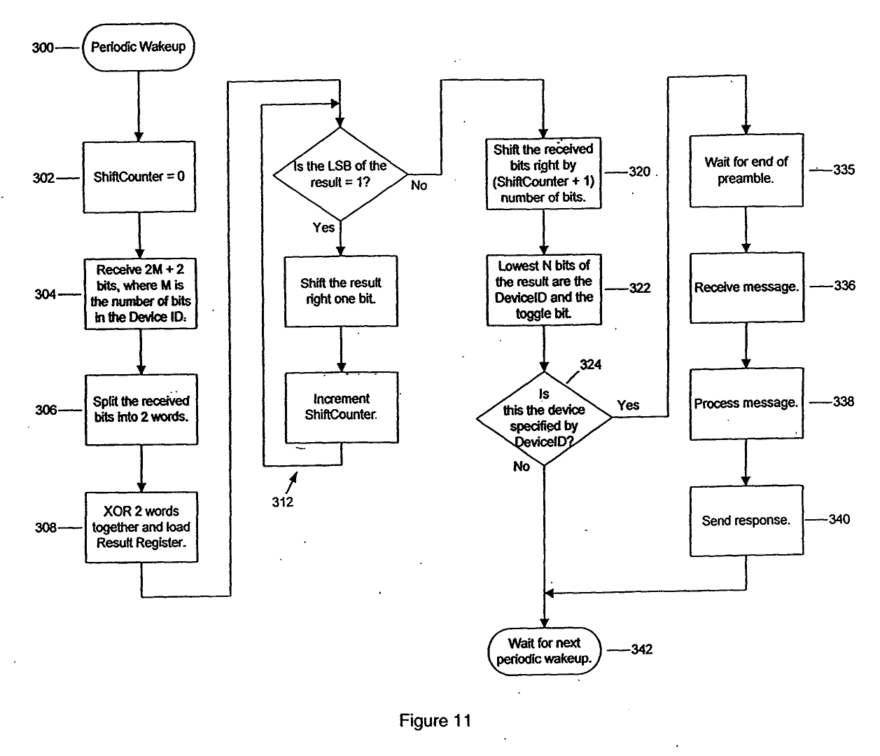

FIG. 11 is a flow chart, similar to FIG. 5, for executing the procedure depicted in FIG. 10. Blocks 310, 302, 304, 306, 308 respectfully correspond to blocks 100, 102, 104, 106, 108, of FIG. 5. Block 308 produces the XOR result of FIG. 10, line C. Loop 312 operates to determine the number of right shifts required to isolate the N bits containing the M bit ID code (FIG. 10, line (F)) and toggle bit block 320 executes the required shifts to extract the M bit ID code. Decision block 324 determines whether an ID code has been extracted. If no, the MD processor 22 causes MD subsystem 18 to resume its sleep state awaiting the next periodic wakeup (block 342). If yes, operation proceeds to block 335 where the processor can stay awake awaiting receipt of the message at the end of the preamble. Blocks 336, 338 and 340 are then executed corresponding to blocks 136, 138 and 15o of FIG. 5.

Although multiple specific embodiments have been described herein, it is recognized that many variations and modifications will occur to those skilled in the art within the spirit of the invention and intended scope of the appended claims.

1. A medical system comprising:
a communication device (CD) including a CD telemetry subsystem selectively operable in a first state to transmit a preamble signal or a second state to transmit a message signal and wherein said preamble signal includes information predicting the time duration to the next message signal; and
a medical device (MD) including an MD telemetry subsystem operable in either a signal receiving state or a low energy consuming state and wherein said MD telemetry subsystem in said signal receiving state is responsive to said information predicting time duration for switching to said low energy consuming state for a time interval related to said predicted time duration.

2. The system of claim 1 wherein said MD telemetry subsystem upon expiration of said time interval switches to said signal receiving state to receive said message signal.

3. The system of claim 1 wherein said medical device is configured for implantation in a patient's body; and wherein
said communication device is configured for use external to said patient's body.

4. The system of claim 3 wherein said medical device includes a programmable MD processor and a therapeutic subsystem controllable by said MD processor for administering therapy to said patient's body; and wherein
said MD telemetry subsystem upon expiration of said time interval switches to said signal receiving state for receiving said message signal; and wherein
said received message signal operates to program said MD processor.

5. The system of claim 1 wherein said preamble signal comprises a serial bit stream defining a sequence of multiple words.

6. The system of claim 5 wherein said medical device further includes an MD processor for processing said preamble signal words to derive said predicted time duration therefrom; and wherein
said MD processor is operable to switch said MD telemetry subsystem to said low energy consuming state for said time interval.

7. The system of claim 6 wherein said MD processor is operable to switch said MD telemetry subsystem to said signal receiving state upon expiration of said time interval.

8. The system of claim 6 wherein said medical device is configured for implantation in a patient's body; and wherein
said communication device is configured for use external to said patients body.

9. The system of claim 5 wherein each preamble word is comprised of an ordered sequence of N bits; and wherein
a corresponding bit in each word defines a constant binary state.

10. The system of claim 5 wherein each preamble word is comprised of an ordered sequence of N bits; and wherein
a corresponding bit in each word toggles between first and second binary states for each successive word.

11. The system of claim 5 wherein each preamble word is comprised of an ordered sequence of N bits including a most significant bit and a least significant bit; and wherein
the most significant bit in each of said words defines a constant binary value and the least significant bit in each of said words toggles between first and second binary values for each successive word.

12. The system of claim 11 including means for performing an exclusive-OR operation with respect to successive words to produce a most significant bit result having a first binary value and a least significant bit result having a second binary value.

13. The system of claim 11 further including means for recognizing the transition between said most significant bit result and said least significant bit result.

14. The system of claim 1 wherein said preamble signal comprises a monotonic sequence of count values where each value defines the time duration to the next message signal.

15. The system of claim 14 wherein said monotonic sequence is comprised of decrementing count values.

16. A method of operating a battery powered implantable medical device, responsive to RF message signals from an external communication device, so as to minimize power consumption in said medical device, said method comprising:
configuring said medical device so that it normally operates in a low energy consuming state and periodically switches to a signal receiving state for a limited duration listening window;
operating said communication device to transmit an RF preamble signal prior to transmission of each RF message signal;
encoding in said preamble signal information predicting the time duration to the next message signal;
causing said medical device during a signal receiving state to decode said predicting information to determine the time duration to the next message signal; and
switching said medical device to said low energy consuming state for a time interval related to said determined time duration.

17. A medical system comprising:
At least one medical device (MD) including an MD power source, an MD therapeutic subsystem, and MD telemetry subsystem, and a programmable MD processor for controlling the operation of said MD therapeutic subsystem and/or said MD telemetry subsystem; and
a communication device (CD) including a CD telemetry subsystem selectively operable to transmit a preamble signal to said MD telemetry subsystem or to transmit a message signal to said MD telemetry subsystem, and wherein control information is embedded in said preamble signal for processing by said MD processor to minimize power consumption in said medical device.

18. The system of claim 17 wherein said embedded control information includes an identification identifying at least one of said medical devices.

19. The system of claim 17 wherein said embedded control information includes time information predicting the time duration to the next message signal.

20. The system of claim 19 wherein said MD telemetry subsystem is operable in either a sleep low energy consumptive state or an awake high energy consumptive state; and wherein
said medical device is responsive to said time information for causing said MD telemetry subsystem to be in said sleep state for the predicted time duration to the next message signal.

21. The system of claim 17 wherein said preamble signal comprises a bit stream comprised of successive N bit words; and wherein
a first fixedly positioned bit in each of said N bit words defines a constant binary value and a second fixedly positioned bit in each of said N bit words comprises a toggle bit whose binary value switches for each successive word.

22. The system of claim 21 including means for performing an exclusive OR operation with respect to each pair of successive words to produce an XOR result; and
means for processing said XOR result to determine word boundaries in said bit stream.
